(19) United States
(12) Patent Application Publication
(10) Pub. No.: US 2026/0034287 A1
(43) Pub. Date: Feb. 5, 2026

(54) IN-LINE DIALYSIS FLUID PREPARATION

(71) Applicant: Mozarc Medical US LLC, Minneapolis, MN (US)

(72) Inventors: Andrea Veratti, Mirandola (IT); Mariachiara Ricci, Mirandola (IT); Stefania Rinaldi, Mirandola (IT); Michele Passerini, Mirandola (IT); Claudio Marella, Mirandola (IT); Roberta Leonardi, Mirandola (IT)

(21) Appl. No.: 19/285,842

(22) Filed: Jul. 30, 2025

Related U.S. Application Data
(60) Provisional application No. 63/678,389, filed on Aug. 1, 2024.
(60) Provisional application No. 63/797,444, filed on Apr. 30, 2025.

(51) Int. Cl. A61M 1/28; A61M 1/14

(52) U.S. Cl. CPC A61M 1/287; A61M 1/154; A61M 1/155; A61M 1/159; A61M 2205/3334; A61M 2205/3368; A61M 2205/3393

(57) ABSTRACT

A system includes a fluid conduit to receive water from a water source, a first and second dialysis fluid concentrate. The system includes a pump fluidly connected to the fluid conduit and to pump the first and second dialysis fluid concentrate. The system includes a first and second dialysis fluid concentrate source fluidly connected to the pump and configured to store the first and second dialysis fluid concentrate, respectively. The system includes a processor configured to control an amount of mixing of the first dialysis fluid concentrate, the second dialysis fluid concentrate, and the water to output a dialysis fluid having a defined composition. The processor determines the defined composition of the dialysis fluid based on a change in weight of the first dialysis fluid concentrate source, the second dialysis fluid concentrate source, a mixing container configured to accommodate the dialysis fluid, or any combination thereof.

### CROSS REFERENCE

This application claims the benefit of and priority to U.S. Provisional Patent Application No. 63/797,444 filed Apr. 30, 2025, and U.S. Provisional Patent Application No. 63/678,389 filed Aug. 1, 2024, the entire content of each of which is incorporated by reference herein.

### FIELD

This disclosure relates generally to systems and methods for producing dialysis fluid.

### BACKGROUND

Dialysis systems can be used to treat patients with kidney disorders. There are a number of dialysis systems in use in the health care industry. Dialysis fluids that are specifically controlled for the dialysis systems are used in these dialysis systems for treatment of the patients.

### SUMMARY

In some embodiments, a system includes a fluid conduit configured to receive water from a water source, a first dialysis fluid concentrate, and a second dialysis fluid concentrate. In some embodiments, the system includes a pump fluidly connected to the fluid conduit and configured to pump the first dialysis fluid concentrate and the second dialysis fluid concentrate. In some embodiments, the system includes a first dialysis fluid concentrate source fluidly connected to the pump and configured to store the first dialysis fluid concentrate. In some embodiments, the system includes a second dialysis fluid concentrate source fluidly connected to the pump and configured to store the second dialysis fluid concentrate. In some embodiments, the system includes a processor configured to control an amount of mixing of the first dialysis fluid concentrate, the second dialysis fluid concentrate, and the water to output a dialysis fluid having a defined composition. In some embodiments, the processor determines the defined composition of the dialysis fluid based on a change in weight of the first dialysis fluid concentrate source, the second dialysis fluid concentrate source, a mixing container configured to accommodate the dialysis fluid, or any combination thereof.

In some embodiments, the first dialysis fluid concentrate is a liquid concentrate.

In some embodiments, the second dialysis fluid concentrate is a liquid concentrate.

In some embodiments, the first dialysis fluid concentrate source is fluidly connected to a water source to receive water to be added to the first dialysis fluid concentrate source.

In some embodiments, the second dialysis fluid concentrate source is fluidly connected to a water source to receive water to be added to the second dialysis fluid concentrate source.

In some embodiments, the system includes a first sensor configured to determine a first amount of the first dialysis fluid concentrate in the first dialysis fluid concentrate source and a second sensor configured to determine a second amount of the second dialysis fluid concentrate in the second dialysis fluid concentrate source.

In some embodiments, at least one of the first sensor or the second sensor is a scale configured to measure a weight.

In some embodiments, the system includes a fluid outlet such that the fluid outlet is configured to receive the dialysis fluid from the mixing container.

In some embodiments, the system includes a sensor upstream of the fluid outlet such that the sensor is configured to determine whether the dialysis fluid meets the defined composition.

In some embodiments, a method includes receiving, in a mixing container, a first dialysis fluid concentrate such that an amount of the first dialysis fluid concentrate received by the mixing container is determined by a change in weight of at least one of: a first dialysis fluid concentrate source, the mixing container, or any combination thereof. In some embodiments, the method includes receiving in the mixing container, a second dialysis fluid concentrate, such that an amount of the second dialysis fluid concentrate received by the mixing container is determined by a change in weight of at least one of: a second dialysis fluid concentrate source, the mixing container, or any combination thereof. In some embodiments, the method includes receiving, in the mixing container, water, such that an amount of the water received by the mixing container is determined by a change in weight of at least one of: the mixing container; the water source container, or any combination thereof.

In some embodiments, the change in weight of at least one of: the first dialysis fluid concentrate source, the mixing container, or any combination thereof, is measured by at least one scale configured to measure the weight of the dialysis fluid concentrate source, the mixing container, or any combination thereof.

In some embodiments, the change in weight of at least one of the second dialysis fluid concentrate source, the mixing container, or any combination thereof, is measured by at least one scale configured to measure the weight of the second fluid concentrate source, the mixing container, or any combination thereof.

In some embodiments, the change in weight of the mixing container is measured by at least one scale configured to measure the weight of the mixing container.

In some embodiments, the method includes determining, by a controller, a composition of a fluid within the mixing container.

In some embodiments, the controller is configured to determine the composition of the fluid within the mixing container based on a change in weight of at least one of: the first dialysis fluid concentrate source, the second dialysis fluid concentrate source, the mixing container, or any combination thereof.

In some embodiments, a method includes receiving, in a mixing container, a first dialysis fluid concentrate and a second dialysis fluid concentrate from at least one fluid concentrate source, such that at least an amount of the first dialysis fluid concentrate and the second dialysis fluid concentrate source are received by the mixing container simultaneously. In some embodiments, an amount of the first dialysis fluid concentrate received by the mixing container is determined by a change in weight of at least one of: a first dialysis fluid concentrate source, the mixing container, or any combination thereof. In some embodiments, an amount of the second dialysis fluid concentrate received by the mixing container is determined by a change in weight of at least one of a second dialysis fluid concentrate source, the mixing container, or any combination thereof. In some embodiments, receiving, in the mixing container, water, such that an amount of the water received by the mixing container is at a period of time subsequent to the mixing container receiving the first dialysis fluid concentrate and the second dialysis fluid concentrate, such that an amount of the water received by the mixing container is determined by a change in weight of the mixing container.

In some embodiments, the change in weight of at least one of the first dialysis fluid concentrate source, the mixing container, or any combination thereof, is measured by at least one scale configured to measure the weight of the dialysis fluid concentrate source, the mixing container, or any combination thereof.

In some embodiments, the change in weight of at least one of: the second dialysis fluid concentrate source, the mixing container, or any combination thereof, is measured by at least one scale configured to measure the weight of the second fluid concentrate source, the mixing container, or any combination thereof.

In some embodiments, the method includes determining, by a controller, a composition of a fluid within the mixing container.

In some embodiments, in the determining, the controller is configured to determine the composition of the fluid within the mixing container based on a change in weight of at least one of the first dialysis fluid concentrate source, the second dialysis fluid concentrate source, the mixing container, or any combination thereof.

### BRIEF DESCRIPTION

References are made to the accompanying drawings that form a part of this disclosure and that illustrate embodiments in which the systems and methods described in this Specification can be practiced.

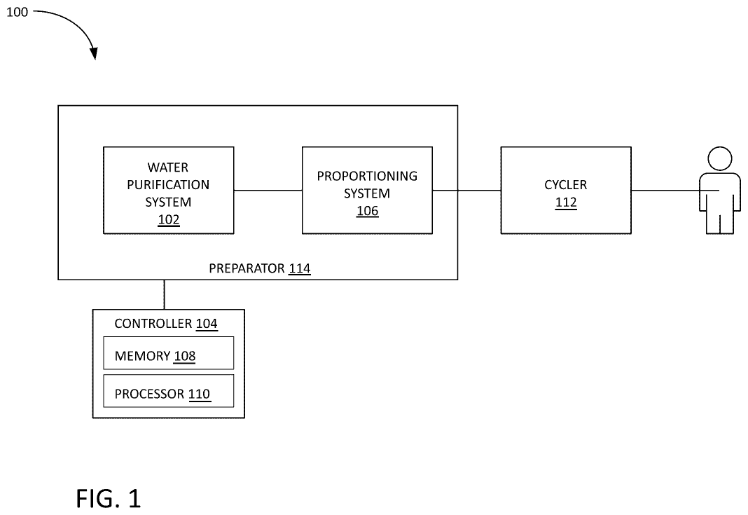

FIG. 1 is a schematic diagram of a dialysis system, according to some embodiments.

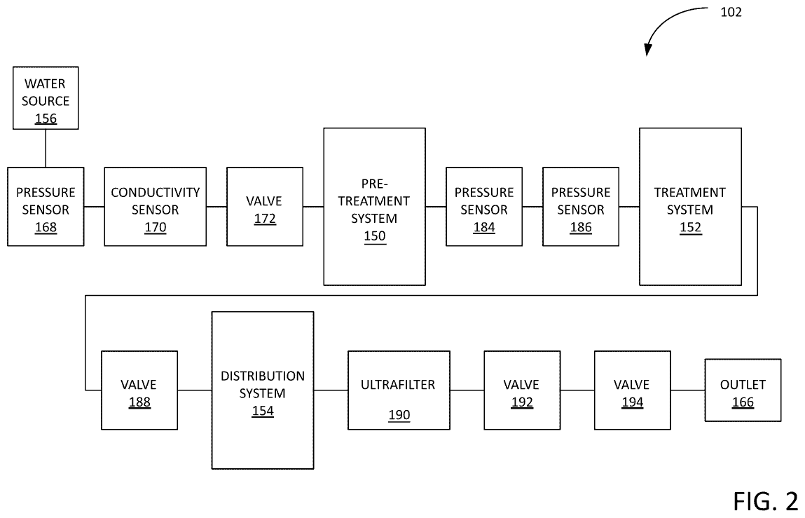

FIG. 2 is a schematic diagram of a water purification system, according to some embodiments.

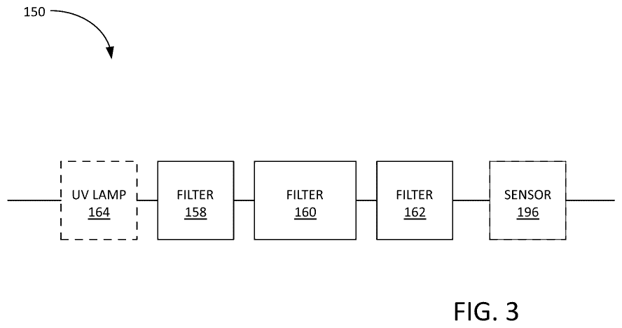

FIG. 3 is a schematic diagram of a pre-treatment system for the water purification system of FIG. 2, according to some embodiments.

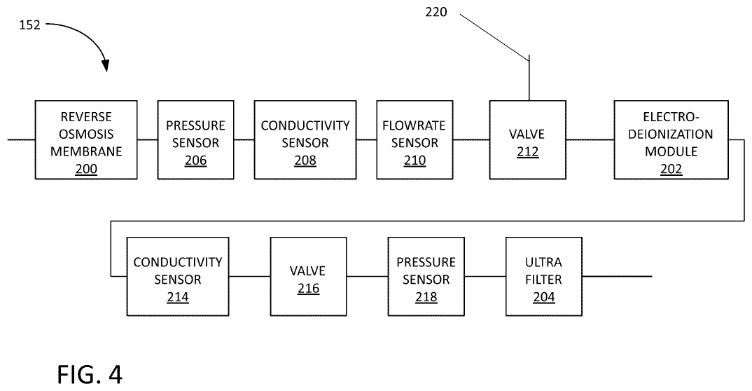

FIG. 4 is a schematic diagram of a treatment system for the water purification system of FIG. 2, according to some embodiments.

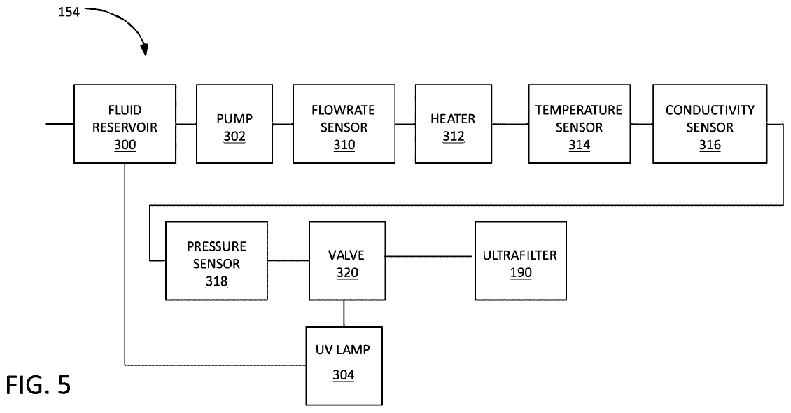

FIG. 5 is a schematic diagram of a distribution system for the water purification system of FIG. 2, according to some embodiments.

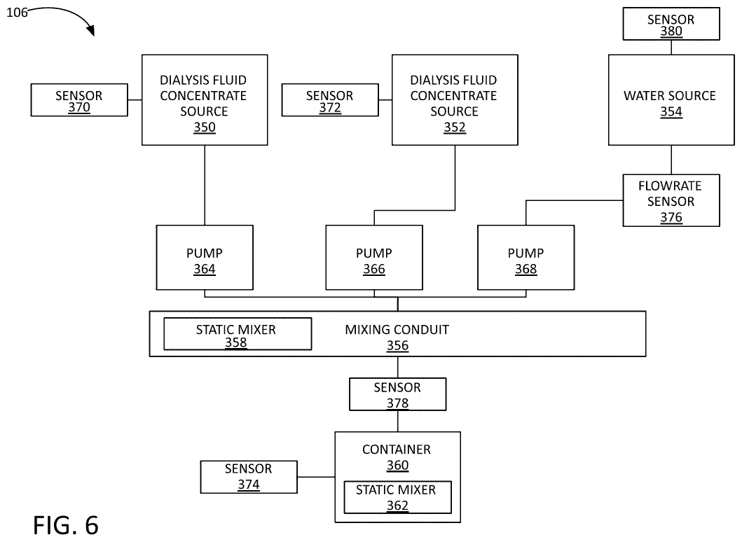

FIG. 6 is a schematic diagram of a proportioning system, according to some embodiments.

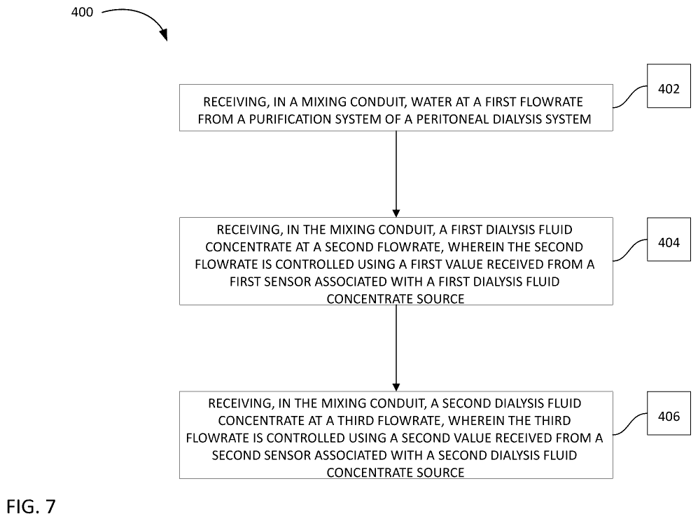

FIG. 7 is a flowchart of a method for preparing dialysis fluid using a proportioning system, according to some embodiments.

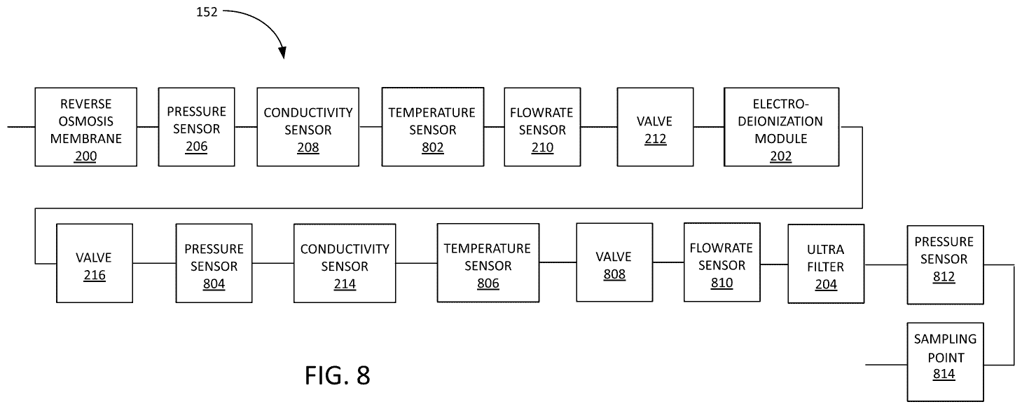

FIG. 8 is a schematic diagram illustrating the treatment system for water purification in a dialysis system, according to some embodiments.

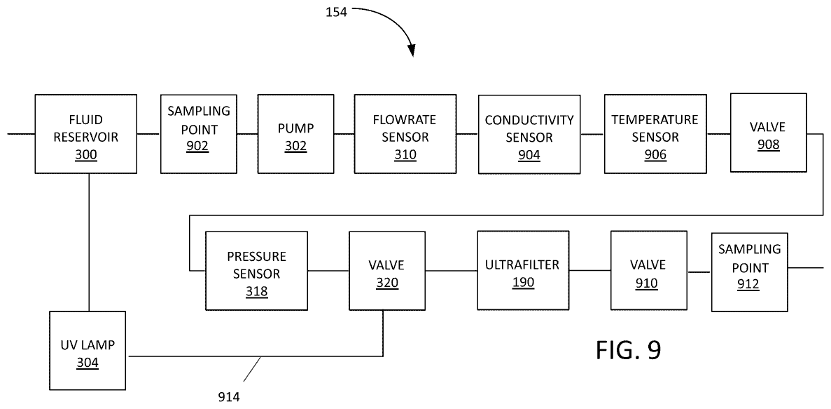

FIG. 9 is a schematic diagram of a distribution system for a water purification system, according to some embodiments.

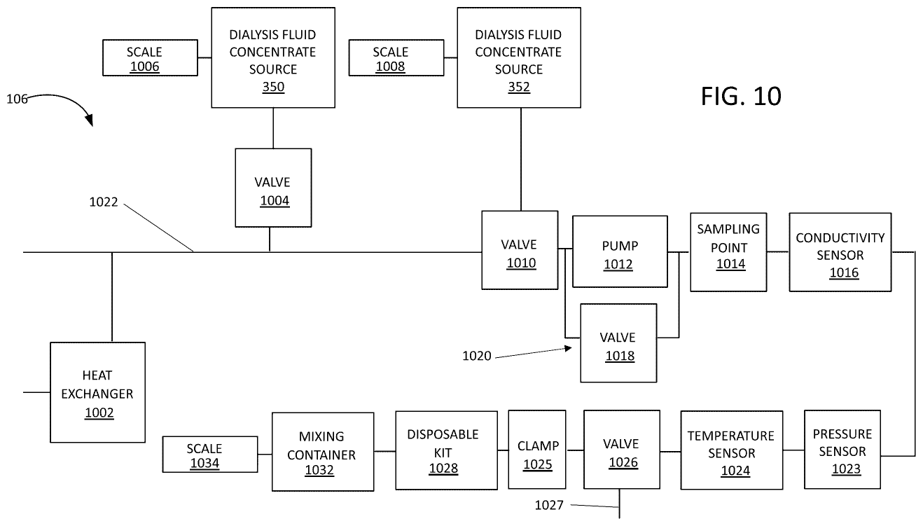

FIG. 10 is a schematic diagram illustrating the components and flow of a proportioning system, according to some embodiments.

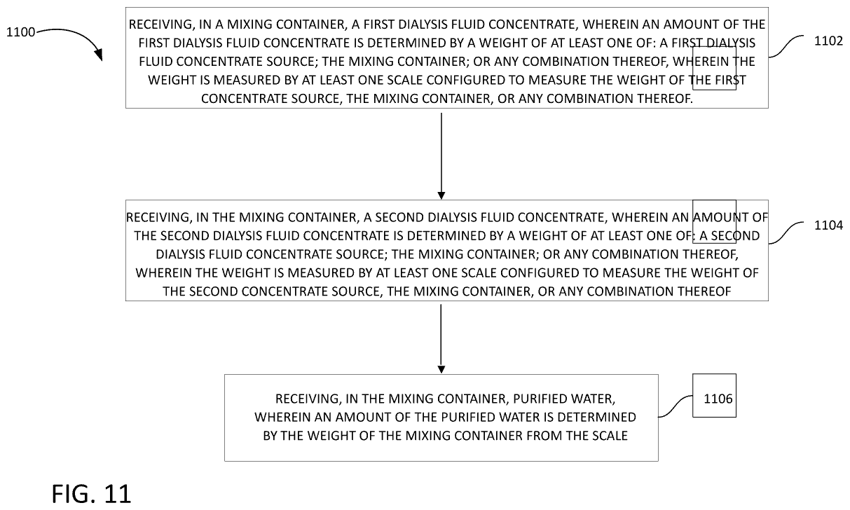

FIG. 11 is a flowchart of a method for preparing dialysis fluid using a proportioning system, according to some embodiments.

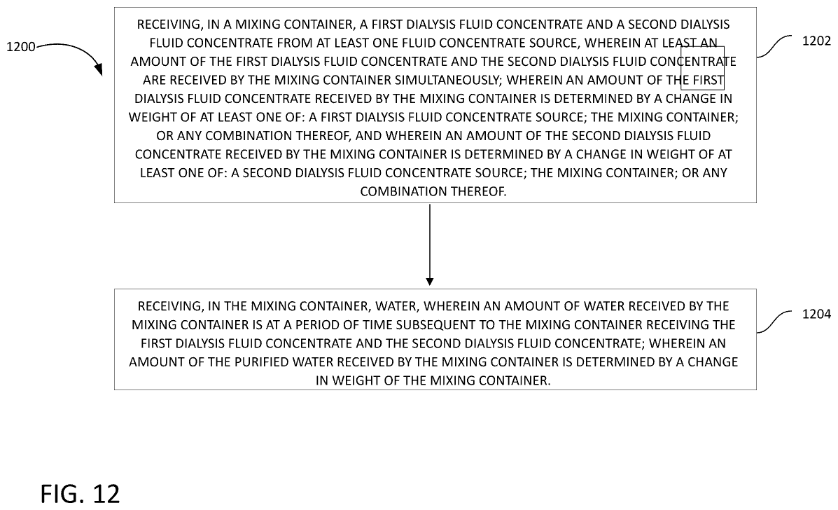

FIG. 12 is a flowchart of a method for preparing dialysis fluid using a proportioning system, according to some embodiments.

### DETAILED DESCRIPTION

Systems and methods for producing dialysis fluid are provided herein, among other things. Dialysis systems can include, for example and without limitation, systems for hemodialysis, hemofiltration, hemodiafiltration, peritoneal dialysis, and the like. To achieve purity levels necessary for patient dialysis treatments, a water source capable of delivering high volumes of water can be required. As one example, the water source can be tap water at households, among other water sources as provided herein. The water purification systems disclosed herein are useful for removing sufficient amounts of contaminants from various water sources and for ensuring the water, once purified, has a composition appropriate for use in preparing dialysis fluids.

Embodiments of this disclosure are directed to improved systems and methods for preparing dialysis fluids. In some embodiments, one or more pumps are configured to pump purified water and dialysis fluid concentrates to a mixing container, where both are mixed or otherwise combined into a dialysis fluid. In some embodiments, the one or more pumps can be controlled based on load cell sensors configured to weigh the dialysis fluid concentrates and the container storing the mixed dialysis fluid. In some embodiments, when the mixed dialysis fluid does not meet parameters of a defined dialysis fluid, the mixture can be drained from the system and prevented from being used in dialysis therapies.

FIG. 1 is a schematic diagram of a dialysis system 100, according to some embodiments. In some embodiments, the dialysis system 100 can be representative of a peritoneal dialysis system including point of use dialysis fluid production. Peritoneal dialysis systems are one example of a dialysis system. It is to be appreciated that the systems and methods described in this disclosure can be applied to other dialysis systems such as, but not limited to, hemodialysis, hemofiltration, hemodiafiltration, or the like.

The illustrated embodiment includes a water purification system 102. A controller 104 is configured to be in electronic communication with a preparator 114, a water purification system 102, a proportioning system 106, or any combination thereof, to send and receive communications relating to sensed parameters, control of valves, or the like. In some embodiments, the water purification system 102 and the proportioning system 106 can be referred to as the preparator 114. The water purification system 102 can be fluidly connected to a proportioning system 106. In some embodiments, the proportioning system 106 can be fluidly connected to a cycler 112. The proportioning system 106 can be configured to prepare a fresh dialysis fluid using purified water output from the water purification system 102. The cycler 112 can receive the fresh dialysis fluid from the proportioning system 106 and can be fluidly connected with a patient to perform the dialysis treatments. The controller 104 can be in electronic communication with the cycler 112 to accomplish the necessary treatments for the patient. It is to be appreciated that the cycler 112 can include one or more additional features such as, but not limited to, a user interface configured to receive user inputs, display outputs for the user, or any combination thereof. In some embodiments, the cycler 112 can have a separate controller from the controller 104.

The controller 104 can be in wired or wireless communication with the water purification system 102, the proportioning system 106, the cycler 112, or any combination thereof. The controller 104 can include a memory 108 and at least one processor 110. It is to be appreciated that the controller 104 can include one or more additional features such as, but not limited to, a display with a user interface configured to receive user inputs, display outputs for the user, or any combination thereof. In some embodiments, a separate user input can also be included so the user can interact with the dialysis system 100.

FIG. 2 is a schematic diagram of the water purification system 102, according to some embodiments. In some embodiments, the water purification system 102 can be broken down into subsystems including a pre-treatment system 150, a treatment system 152, and a distribution system 154. In some embodiments, the water purification system 102 can be contained within an apparatus that is fluidly connected to the cycler 112.

The water purification system 102 is fluidly connected to a water source 156. For example, the water source 156 can be a water tap or the like. The fluid received at the water purification system 102 from the water source 156 can be treated using the pre-treatment system 150, the treatment system 152, and the distribution system 154.

In some embodiments, the distribution system 154 includes an outlet 166 configured to be fluidly connected to the proportioning system 106.

In some embodiments, one or more additional components can be included in the water purification system 102. For example, the water purification system 102 can include a pressure sensor 168, a conductivity sensor 170, and a valve 172 fluidly disposed between the water source 156 and the pre-treatment system 150. In some embodiments, a pressure sensor 184 and a pressure sensor 186 can be disposed fluidly between the pre-treatment system 150 and the treatment system 152. In some embodiments, a valve 188 can be disposed fluidly between the treatment system 152 and the distribution system 154. In some embodiments, an ultrafilter 190, a valve 192, and a valve 194 can be disposed between the distribution system 154 and the cycler 112 (FIG. 1). In some embodiments, the ultrafilter 190 can be included in the distribution system 154.

In some embodiments, the valve 172, the valve 188, the valve 192, and the valve 194 can be electronically controlled valves in fluid communication with the controller 104 of the water purification system 102. In some embodiments, the valve 172, the valve 188, the valve 192, and the valve 194 can be selectively activated to drain the water from the water purification system 102. As such, although not shown in the figure, the valve 172, the valve 188, the valve 192, and the valve 194 can also be fluidly connected with a drain of the water purification system 102.

In some embodiments, the controller 104 can be configured to receive inputs from the pressure sensor 168, the conductivity sensor 170, the pressure sensor 184, and the pressure sensor 186 (in addition to other sensors shown and described in additional detail in FIGS. 3-5 below) to selectively drain water from the water purification system 102 via opening of one or more of the valve 172, the valve 188, the valve 192, and the valve 194. For example, in some embodiments, if a condition is detected that indicates that one or more parameters of the water are not being met by the pre-treatment system 150, the treatment system 152, or the distribution system 154, the controller 104 can selectively open one of the valves to ensure that water not meeting purity requirements is not output to proportioning system 106.

In some embodiments, by being able to open a variety of valves for this purpose, it is possible to prevent the water from unnecessarily going through components of the water purification system 102, which can prolong a lifetime of those components when an upstream failure occurs. Additionally, in this manner, it is possible to maintain a required purity of the water during the course of filtration. In some embodiments, this can provide a real-time understanding of whether the water meets the purity requirements for the dialysis system 100.

FIG. 3 is a schematic diagram of the pre-treatment system 150 for the water purification system 102, according to some embodiments. In some embodiments, the pre-treatment system 150 can be configured to reduce bacteria and sediment, filter coarse particles, reduce hardness, and remove heavy metals from the water received via the water source 156.

In some embodiments, the pre-treatment system 150 includes a first filter 158, a second filter 160, and a third filter 162 connected in series. In some embodiments, the pre-treatment system 150 can additionally include an ultraviolet (UV) lamp 164 to disinfect the water stream. In some embodiments, the lamp 164 can be included instead of the first filter 158.

In some embodiments, the second filter 160 and the third filter 162 can be the same filters. For example, the first filter 158, the second filter 160, and the third filter 162 can include a sediment filter, a softener filter, an activated carbon filter, or any combination thereof. In some embodiments, the sediment filter can remove particulates from the fluid, the softener filter can remove minerals from hard fluid, and an activated carbon filter can remove certain chemicals from the fluid. In some embodiments, the third filter 162 can be a similar type of filter to the second filter 160. For example, in some embodiments, the third filter 162 and the second filter 160 can both be activated carbon filters thereby acting as a filtration redundancy.

In some embodiments, the second filter 160, the third filter 162, or both the second filter 160 and the third filter 162 can be an activated carbon filter. In some embodiments, the second filter 160 and the third filter 162 can reduce a concentration of chlorine and chloramine in the water. In some embodiments, the third filter 162 can serve to act in case of a failure by the second filter 160.

In some embodiments, the third filter 162 can be used to remove endotoxins from the water. In some embodiments, the lamp 164 can be used to kill bacteria in the water.

In some embodiments, the first filter 158, the second filter 160, and the third filter 162 can be configured to collectively remove particles from the water. In some embodiments, the particles being removed can include clay, silt, silicon, combinations thereof, or the like.

In some embodiments, the first filter 158, the second filter 160, and the third filter 162 can be configured to collectively remove chlorine and compositions including chlorine from the water. In some embodiments, the first filter 158, the second filter 160, and the third filter 162 can be configured to collectively absorb toxic substances such as, but not limited to, pesticides. In some embodiments, the first filter 158, the second filter 160, and the third filter 162 can be configured to collectively remove hypochlorite, chloramine, and chlorine from the water.

In some embodiments, the first filter 158, the second filter 160, and the third filter 162 can include a sediment filter, a softener filter, at least one activated carbon filter, or any combination thereof. In some embodiments, a UV lamp can be disposed between the softener filter and the activated carbon filters of the first filter 158, second filter 160, and the third filter 162.

In some embodiments, the pre-treatment system 150 can additionally include a sensor 196 disposed downstream of the third filter 162. In some embodiments, the sensor 196 can be used to assess performance of the second filter 160 and the third filter 162. In some embodiments, the sensor 196 can provide an estimate of levels of contamination in the water exiting the water purification system 102. In some embodiments, the controller 104 (FIG. 2) can be configured to change a state of the valve 172 (FIG. 2) if the reading from the sensor 196 is greater than a threshold value. In some embodiments, being greater than the threshold value can be an indication that water purification is not reaching required purity levels. It is to be appreciated that the sensor 196 may not directly identify which contaminants are passing through the pre-treatment system 150 but can give an indication that one or both of the second filter 160 and the third filter 162 are not working effectively.

In some embodiments, causing a drain of the system can also include providing an output from the controller 104 (FIG. 2) to generate an alert to indicate that the water purification system 102 is not working properly and may need to be serviced.

FIG. 4 is a schematic diagram of the treatment system 152 for the water purification system 102, according to some embodiments. In some embodiments, the treatment system 152 can include a reverse osmosis membrane 200, an electro-deionization module 202, and an ultrafilter 204.

In some embodiments, the treatment system 152 includes one or more additional components. In some embodiments, the treatment system 152 can include a pressure sensor 206, a conductivity sensor 208, a flowrate sensor 210, and a valve 212 fluidly disposed downstream of the reverse osmosis membrane 200 and upstream of the electro-deionization module 202.

In some embodiments, a conductivity sensor 214, a valve 216, and a pressure sensor 218 can be disposed fluidly downstream of the electro-deionization module 202 and fluidly upstream of the ultrafilter 204.

In some embodiments, the valve 188 (FIG. 2) can be disposed fluidly downstream of the ultrafilter 204.

In some embodiments, the controller 104 can be configured to monitor at least one of the pressure sensor 206, conductivity sensor 208, and flowrate sensor 210. In some embodiments, monitoring these components of the treatment system 152 can provide an understanding of whether the reverse osmosis membrane 200 is functioning properly. In some embodiments, if, for example, aluminum is passing through the reverse osmosis membrane 200, a conductivity measured by the conductivity sensor 208 would be higher than if the reverse osmosis membrane 200 is functioning properly. In such embodiments, an efficiency of the reverse osmosis membrane 200 may be reduced compared to a properly functioning reverse osmosis membrane. In some embodiments, the conductivity sensor 208 can accordingly be used to infer whether the treatment system 152 is properly removing metals such as, but not limited to, aluminum. In some embodiments, if the conductivity as measured by the conductivity sensor 208 is higher than a threshold conductivity, the controller 104 (FIG. 1) can be configured to change a state of the valve 212 to drain the water from the water purification system 102 to a drain 220 and prevent water from continuing through the treatment system 152. In some embodiments, the controller 104 (FIG. 1) can be configured to change a state of the valve 172 (FIG. 2) instead of, or in addition to, the valve 212 to prevent water from depleting the filters in the pre-treatment system 150 (FIG. 2) if a suspected problem is identified with the reverse osmosis membrane 200. In some embodiments, the controller 104 (FIG. 1) may make the decision based on a combination of the readings from the pressure sensor 206, the conductivity sensor 208 and the flowrate sensor 210.

In some embodiments, the controller 104 (FIG. 1) can be configured to monitor the conductivity sensor 214. In some embodiments, like the reverse osmosis membrane 200, the electro-deionization module 202 is configured to remove metals such as, but not limited to, aluminum from the water. In some embodiments, if the conductivity as measured at conductivity sensor 214 is higher than a threshold value, then it can be inferred that more metal content is passing through the electro-deionization module 202 than desired. As a result, the controller 104 (FIG. 1) can be configured to open the valve 212 and drain the water from the water purification system 102. In some embodiments, the controller 104 (FIG. 1) can be configured to change a state of the valve 172 (FIG. 2) instead of, or in addition to, the valve 212 to prevent water from depleting the filters in the pre-treatment system 150 if a suspected problem is identified with the electro-deionization module 202.

In some embodiments, the reverse osmosis membrane 200 and the electro-deionization module 202 can generally be configured to control a concentration of nitrates in the water being filtered. In some embodiments, if the concentration of nitrates in the water is higher than desired, the conductivity will also be higher than expected. As a result, readings from the conductivity sensor 208 can be used to infer whether the reverse osmosis membrane 200 is properly functioning and removing nitrates as expected. If the conductivity is higher than a threshold value, the controller 104 can be configured to change a state of the valve 212 to drain the water from the water purification system 102 and prevent water from continuing through the treatment system 152. In some embodiments, the controller 104 (FIG. 1) can be configured to change a state of the valve 172 (FIG. 2) instead of, or in addition to, the valve 212 to prevent water from depleting the filters in the pre-treatment system 150 (FIG. 2) if a suspected problem is identified with the reverse osmosis membrane 200.

In some embodiments, the controller 104 (FIG. 1) can be configured to monitor the conductivity sensor 214. In some embodiments, like the reverse osmosis membrane 200, the electro-deionization module 202 is configured to remove nitrates from the water being filtered. In some embodiments, if the conductivity as measured at conductivity sensor 214 is higher than a threshold value, then it can be inferred that more nitrates are passing through the electro-deionization module 202 than desired. As a result, the controller 104 (FIG. 1) can be configured to open the valve 212 and drain the water from the water purification system 102. In some embodiments, the controller 104 (FIG. 1) can be configured to change a state of the valve 172 (FIG. 2) instead of, or in addition to, the valve 212 to prevent water from depleting the filters in the pre-treatment system 150 if a suspected problem is identified with the electro-deionization module 202.

FIG. 5 is a schematic diagram of the distribution system 154 for the water purification system 102, according to some embodiments. In some embodiments, the distribution loop can include a fluid reservoir 300 and a pump 302 that is configured to circulate the water within the distribution system 154 to prevent stagnation. In some embodiments, the distribution system 154 includes a UV lamp 304. In some embodiments, the UV lamp 304 can prevent bacterial formation. In some embodiments, the ultrafilter 190 (FIG. 2) is configured to be located downstream of the distribution system 154 and upstream of the proportioning system 106, the cycler 112 (FIG. 1), or both, to remove endotoxins produced by the UV lamp 304. In some embodiments, the ultrafilter 190 can be disposed outside of the distribution system 154.

In some embodiments, the distribution system 154 includes the pump 302, a flowrate sensor 310, a heater 312, temperature sensor 314, a conductivity sensor 316, a pressure sensor 318, and a valve 320. In some embodiments, the valve 320 can be controlled to enable the water to either recirculate to the fluid reservoir 300 or to be provided to the ultrafilter 190. In some embodiments, the valve 192 (FIG. 2) is disposed downstream of the ultrafilter 190 to drain water if necessary. In some embodiments, the valve 194 (FIG. 2) is also disposed downstream of the ultrafilter 190 (FIG. 2) to control whether purified water is provided from the outlet 166 (FIG. 2) to the cycler 112 (FIG. 1).

With reference to FIGS. 2-5 collectively, in some embodiments, the water purification system 102 is configured to control ion removal from the source water. In some embodiments, the conductivity sensors (conductivity sensor 170, conductivity sensor 208, conductivity sensor 214, and conductivity sensor 316) can be used to assess whether the filtration steps in pre-treatment system 150, treatment system 152, and distribution system 154 are working properly. At conductivity sensor 170, the conductivity of the source water is determined. At conductivity sensor 208, the conductivity of the water downstream of the reverse osmosis membrane 200 is determined. At conductivity sensor 214, the conductivity of the water downstream of the electro-deionization module 202 is determined. At conductivity sensor 316, the conductivity of the water in the distribution loop is determined. At each of these locations, the conductivity of the water should be trending downward. If at any of the locations downstream of the conductivity sensor 170 the conductivity is not decreasing, this can indicate a problem in the system and that ions are not being properly removed from the water. In some embodiments, the controller 104 can control one or more of the valve 172, the valve 188, the valve 192, the valve 194, the valve 212, the valve 216, or the valve 320 to drain the water from the system. As discussed above, the location of the valve being opened will be selected by the controller 104 to prevent unnecessary usage of the components of the water purification system 102 when an error condition has been identified.

FIG. 6 is a schematic diagram of the proportioning system 106 (FIG. 1), according to some embodiments.

In some embodiments, the proportioning system 106 includes a dialysis fluid concentrate source 350 and a dialysis fluid concentrate source 352. In some embodiments, it could be possible for the dialysis fluid concentrate source 350 and the dialysis fluid concentrate source 352 to be pre-mixed in a single container. In some embodiments, the dialysis fluid concentrate source 350 can include an ion solution. In some embodiments, the dialysis fluid concentrate source 352 can include a dextrose solution. In some embodiments, a third dialysis fluid concentrate source can be included. In such embodiments, the third dialysis fluid concentrate source can include bicarbonate. In some embodiments, the bicarbonate can be combined with either the dialysis fluid concentrate source 350 or the dialysis fluid concentrate source 352. In some embodiments, the dialysis fluid concentrate source 350 can be in a powder form. In some embodiments, the dialysis fluid concentrate source 350 can be in a liquid form. In some embodiments, the dialysis fluid concentrate source 352 can be in a powder form. In some embodiments, the dialysis fluid concentrate source 352 can be in a liquid form. In embodiments in which the dialysis fluid concentrate source 350 or the dialysis fluid concentrate source 352 are in powder form, water can be added to the dialysis fluid concentrate source 350 or the dialysis fluid concentrate source 352 in powder form via a water source 354 prior to being utilized by the proportioning system 106. In some embodiments, the water source 354 can be the water purification system 102 (FIG. 1). For example, the water source 354 can be the fluid reservoir 300. In some embodiments, the water source 354 can be a source separate from the water purification system 102 (FIG. 1) such as, but not limited to, a container such as, but not limited to, a bag or the like.

In some embodiments, the dialysis fluid concentrate source 350 and the dialysis fluid concentrate source 352 can be stored in a container such as, but not limited to, a bag having fluid connections for fluidly connecting to a mixing conduit 356. In some embodiments, the mixing conduit 356 is configured to receive water from the water source 354, dialysis fluid from the dialysis fluid concentrate source 350, and dialysis fluid from the dialysis fluid concentrate source 352. In some embodiments, the mixing conduit 356 is configured to receive the water and the dialysis fluid concentrates at the same time. In some embodiments, the mixing conduit 356 can have a static mixer 358 within the mixing conduit 356. In some embodiments, the mixing conduit 356 can include a plurality of the static mixer 358. For example, in some embodiments, static mixers can be included at junctions of each of the dialysis fluid concentrate conduits with the mixing conduit 356. The static mixer 358 can be a fixed device that is installed within the mixing conduit 356 to allow for the blending of the water and the dialysis fluid concentrates as they move through the mixing conduit 356.

It is to be appreciated that the dialysis fluid concentrate source 350 and the dialysis fluid concentrate source 352 being separate is an example. For example, in some embodiments, the dialysis fluid concentrate source 350 and the dialysis fluid concentrate source 352 can be combined into the same container.

In some embodiments, the fluids received from the dialysis fluid concentrate source 350, the dialysis fluid concentrate source 352, and the water source 354 can be provided via the mixing conduit 356 to a container 360. In some embodiments, the container 360 can be referred to as a mixing container or the like. In some embodiments, the container 360 can be a container such as, but not limited to, a bag or the like.

In some embodiments, the container 360 can include a static mixer 362. In some embodiments, neither the static mixer 358 nor the static mixer 362 are included. In some embodiments, either the static mixer 358 or the static mixer 362 can be included. In some embodiments, both the static mixer 358 and the static mixer 362 can be included.

In some embodiments, the proportioning system 106 includes a pump 364. In some embodiments, the pump 364 can be fluidly connected to the dialysis fluid concentrate source 350, the dialysis fluid concentrate source 352, the water source 354, or combinations thereof. In some embodiments, the proportioning system 106 includes the pump 364.

In some embodiments, the proportioning system 106 includes the pump 368 for pumping water from the water source 354 and a pump 366 for pumping the dialysis fluid concentrate source 350 and the dialysis fluid concentrate source 352.

In some embodiments, the proportioning system 106 includes the pump 368 for pumping water from the water source 354, the pump 364 for pumping the dialysis fluid concentrate source 350, and a pump 366 for pumping the dialysis fluid concentrate source 352.

In some embodiments, the proportioning system 106 can be controlled by the controller 104 (FIG. 1). In some embodiments, the proportioning system 106 can have a separate controller from the controller 104.

In some embodiments, the proportioning system 106 includes a plurality of sensors. In some embodiments, a first sensor 370 is disposed at a location of the dialysis fluid concentrate source 350. In some embodiments, a second sensor 372 is disposed at a location of the dialysis fluid concentrate source 352. In some embodiments, a third sensor 374 is disposed at a location of the container 360. In some embodiments, at least one of the first sensor 370, the second sensor 372, or the third sensor 374 are load cell sensors configured to monitor a weight. For example, in some embodiments, the first sensor 370 is a load cell sensor configured to determine a weight of the dialysis fluid concentrate source 350. In some embodiments, the weight of the dialysis fluid concentrate source 350 can be used to determine an amount of the dialysis fluid concentrate source 350 remaining. In some embodiments, the second sensor 372 is a load cell sensor configured to determine a weight of the dialysis fluid concentrate source 352. In some embodiments, the weight of the dialysis fluid concentrate source 352 can be used to determine an amount of the dialysis fluid concentrate source 352 remaining. In some embodiments, the third sensor 374 is a load cell sensor configured to determine a weight of the mixed dialysis fluid stored in the container 360. In some embodiments, the weight of the mixed dialysis fluid stored in the container 360 can be used to determine an amount of the mixed dialysis fluid that has been formulated.

In some embodiments, in addition to load cell sensors, one or more flowrate sensors 376 can be disposed in the mixing conduit 356. In some embodiments, one or more flowrate sensors 376 can be used by the controller 104 to determine an initial volumetric delivery of the purified water from the water source 354. In some embodiments, determining the weights and corresponding amounts of the fluids using load cell sensors can enable more responsive controls of the pump 364, the pump 366, and the pump 368 to ensure that proper mixing of the fluids is achieved so that the concentration of the dialysis fluid in the container 360 corresponds to a defined composition (within acceptable accuracy limits) that is needed to accomplish the dialysis therapy for the patient. In some embodiments, the pump 364, the pump 366, the pump 368 can be volumetric pumps, with a set rpm, that can be used by the controller 104 to determine the flowrate of solutions from the dialysis fluid concentrate source 350 and the dialysis fluid concentrate source 352.

In some embodiments, when dialysis fluid is needed, the controller 104 (FIG. 1) can withdraw an amount of dialysis fluid concentrate from the dialysis fluid concentrate source 350, an amount of dialysis fluid concentrate from the dialysis fluid concentrate source 352, and an amount of water from the water source 354. In some embodiments, the amounts for each of the dialysis fluid concentrates and the water can be controlled by enabling one or more of the pump 364, the pump 366, or the pump 368 for a period of time and at a set flowrate. In some embodiments, the flowrate can be variable if the pump 364, the pump 366, or the pump 368 are variable speed pumps. In some embodiments, if the pump 364, the pump 366, or the pump 368 are fixed speed pumps, the period of time can be controlled while the flowrate is fixed. In some embodiments, the controller 104 (FIG. 1) can set the period of time, the flowrate, or combination thereof, to achieve a particular volume of the mixed dialysis fluid. In some embodiments, the controller 104 receives feedback from the first sensor 370, the second sensor 372, and the third sensor 374 to control the period of time, the flowrate, or a combination thereof. In some embodiments, the controller 104 can enable one of the pump 364, the pump 366, or the pump 368 and disable the one of the pump 364, the pump 366, or the pump 368 when a target weight is reached, indicating that an expected volume of the corresponding fluid has been dispensed. In some embodiments, the pump 364, the pump 366, and the pump 368 can be enabled simultaneously so that the dialysis fluid concentrates and the water are provided to the mixing conduit 356 at the same time and are received in the container 360 in a mixed form. In some embodiments, the controller 104 (FIG. 1) can use sensed values from the first sensor 370, the second sensor 372, and the third sensor 374 over time to calculate a rate of change of the weights and to predict when the amount of mixed fluid in the container 360 would be expected to be achieved, and accordingly disable the corresponding ones of the pump 364, the pump 366, or the pump 368.

In some embodiments, the proportioning system 106 can include one or more sensors 378 configured to check one or more parameters of the mixed dialysis fluid prior to the mixed dialysis fluid reaching the container 360. In such embodiments, if one of the parameters is identified as being outside of a defined parameter, the controller 104 (FIG. 1) can divert the mixed fluid from the mixing conduit 356 to a drain to prevent the container 360 from receiving fluids that would not be suitable for the dialysis treatment. In some embodiments, in addition to diverting to the drain, the controller 104 (FIG. 1) can disable all of the pump 364, the pump 366, and the pump 368 present in the proportioning system 106 to stop the formulation of additional dialysis fluid. In some embodiments, the controller 104 (FIG. 1) can also generate an alarm that can be audible, visual, or a combination thereof to alert the user that there is a problem with the proportioning system 106.

In some embodiments, the proportioning system 106 can include a sensor 380 at a location of the water source 354. In some embodiments, the sensor 380 can be in place of the third sensor 374. In some embodiments, the sensor 380 can be in addition to the third sensor 374. In some embodiments, the sensor 380 is a load cell sensor configured to monitor a weight. For example, in some embodiments, the sensor 380 is a load cell sensor configured to determine a weight of the water source 354. In some embodiments, the weight of the water source 354 can be used to determine an amount of purified water remaining in the water source 354.

In some embodiments, the proportioning system 106 can include one or more additional filters (e.g., ultrafilters or the like) to ensure the mixed dialysis fluid is sterilized.

FIG. 7 is a flowchart of an example method 400 preparing dialysis fluid using a proportioning system (e.g., the proportioning system 106 of FIG. 6), according to some embodiments.

In some embodiments, at block 402, the method 400 includes receiving, in a mixing conduit (e.g., mixing conduit 356 of FIG. 6), water at a first flowrate from a water source (e.g., water purification system 102 of FIG. 1) of a peritoneal dialysis system (e.g., dialysis system 100 of FIG. 1). In some embodiments, the first flowrate is controlled using a first sensed value received from a first sensor (e.g., sensor 378 of FIG. 6) associated with the container. In some embodiments, the one or more sensors 378 is a flow rate sensor. In some embodiments, the first flowrate is controlled using a first sensed value received from a first sensor (e.g., sensor 380 of FIG. 6) associated with a water source storage.

In some embodiments, at block 404, the method 400 includes receiving, in the mixing conduit 356, a first dialysis fluid concentrate (e.g., from dialysis fluid concentrate source 350 of FIG. 6) at a second flowrate. In some embodiments, the second flowrate is controlled using a first value received from a first sensor (e.g., first sensor 370 of FIG. 6) associated with a first dialysis fluid concentrate source.

At block 406, the method 400 includes receiving, in the mixing conduit 356, a second dialysis fluid concentrate (e.g., from dialysis fluid concentrate source 352 of FIG. 6) at a third flowrate, wherein the third flowrate is controlled using a second value received from a second sensor (e.g., the second sensor 372 of FIG. 6) associated with a second dialysis fluid concentrate source.

FIG. 8 shows, according to some embodiments, a detailed schematic of a treatment system for water purification in a dialysis system, illustrating the integration and interaction of various components to filter water suitable for dialysis treatments. FIG. 8 is a schematic diagram of a treatment system 152. The treatment system 152 for water purification is the same or similar to the system 152 shown in FIG. 4, for water purification. Accordingly, similar reference numbers are used for similar components. Some of the differences between the treatment system 152 shown in FIG. 8 and the treatment system 152 shown in FIG. 4 are discussed. It will, however, be appreciated that other differences may exist, without departing from the scope of this disclosure.

The reverse osmosis membrane 200 can serve, according to some embodiments, as the initial filtration stage of the treatment system 152, effectively removing a wide range of contaminants from the water. The pressure sensor 206 can monitor the pressure downstream from the reverse osmosis membrane 200. This can provide pressure information to the controller 104 regarding the operating conditions and allowing the detection of any potential issues that may affect filtration efficiency. The conductivity sensor 208 measures the ionic content of the water, providing an indication of the membrane's performance in removing dissolved salts or any other impurities found in the liquid as it passes through the reverse osmosis membrane 200. In some embodiments, the reverse osmosis membrane 200 can include at least one proportional valve to permit fluid communication between the reverse osmosis membrane 200 and the drain line to improve the yield of the water purification. In some embodiments, the reverse osmosis membrane 200 can be a double stage reverse osmosis membrane filter. In some embodiments, the reverse osmosis membrane 200 can include more than one reverse osmosis membrane 200. For example, the fluid being filtered can be filtered by two reverse osmosis membranes 200 in sequential stages.

Following the reverse osmosis membrane 200, the temperature sensor 802 is employed to monitor the water temperature, which can influence the efficiency of subsequent filtration processes, or provide the controller 104 information regarding the state of the liquid flow at this point in the filtration process. In some embodiments, the temperature sensor 802 can be used by the controller 104 for a temperature compensation in conductivity measurements. In some embodiments, the temperature sensor 802 can be used to ensure that the operating temperature of an electro-deionization module, for example, the electro-deionization module 202, is within an operable range. For example, the temperature sensor 802 can be used to confirm that the fluid is within about 5° C. to about 35° C. If the temperature sensed by the 802 is determined to be outside of the operable range, it may indicate to the controller 104 that the composition of the water may be outside of requirements.

In some embodiments, the conductivity sensor 208 and the temperature sensor 802 can be combined as a single component. In some embodiments, the conductivity sensor 208 and the temperature sensor 802 can be a single sensor capable of measuring the electrical conductivity of the solution along with the temperature of the solution.

In some embodiments, the treatment system 152 can include the flowrate sensor 210. The flowrate sensor 210 can measure the rate at which water flows through the system. The data gathered by the flowrate sensor 210 can permit the controller 104 precise control of the liquid flow through the reverse osmosis membrane 200 and adjust the flow rate to maintain desired filtration rates. For example, the reverse osmosis membrane 200 can be influenced by the flow rate of the liquid therethrough.

The valve 212 is strategically placed to control the flow of water into the electro-deionization module 202. This module further purifies the water by removing remaining ions through an electrochemical process, enhancing the overall purity of the water.

The pressure sensor 804, the conductivity sensor 214, and the temperature sensor 806, positioned after the electro-deionization module 202, offer real-time monitoring of the water's pressure, ionic content, and temperature respectively. These sensors play an important role in assessing the electro-deionization module's 202 performance. The data from the pressure sensor 804, the conductivity sensor 214, and the temperature sensor 806 can be provided to the controller 104. The controller 104 can analyze the data provided by the pressure sensor 804, the conductivity sensor 214, and the temperature sensor 806 to determine that the water meets the purity requirements necessary for to be mixed in with dialysis fluid. In some embodiments, the conductivity sensor 214 and the temperature sensor 806 can be combined as a single component. In some embodiments, the conductivity sensor 214 and the temperature sensor 806 can be a single sensor capable of measuring the electrical conductivity of the solution along with the temperature of the solution. In some embodiments, the electro-deionization module 202 can include at least one proportional valve to permit fluid communication between the reverse osmosis membrane 200 and the drain line to improve the yield of the water purification.

In some embodiments, a valve 808 is positioned downstream from sensors 804, 214, and 806, so that the fluid, if the controller 104 determines that the filtration requirements are not achieved, can be diverted from proceeding. In some embodiments, the fluid can be diverted to a drain if the controller 104 determines that, based on the measurements from the sensors 214, 804, and 806, the fluid does not meet desired characteristics or has specific impurities that cannot be filtered. In some embodiments, the fluid can be recircled back to a specific point of filtration, as determined by the controller 104. For example, the controller 104 can determine that the fluid needs to be re-filtered by the reverse osmosis membrane 200 and can deliver the fluid upstream of the reverse osmosis membrane 200. The flowrate sensor 810, similar to the flowrate sensor 210, continues to monitor the water flow, permitting the controller 104 to analyze the flow rate data at this point, thereby ensuring consistent delivery of the fluid to the ultrafilter 204.

In some embodiments, the treatment system 152 can include the ultrafilter 204. The ultrafilter 204 can serve as the final filtration stage in the treatment system 152, in some embodiments, removing any remaining particulates and ensuring the water is free from endotoxins and other contaminants. In some embodiments, the ultrafilter 204 can reduce the bacterial and endotoxins load within the fluid. The pressure sensor 812, located downstream of the ultrafilter 204, provides additional monitoring of the pressure at this point and can provide this data to the controller 104. The controller 104 can determine from the provided pressure that the system operates within safe pressure limits.

The treatment system 152 can include a sampling point 814 for periodic testing and verification of the water quality. In some embodiments, this data can be transmitted to the controller 104. Through analysis of the data, the controller 104 can determine if the water meets the required standards for dialysis treatments. This comprehensive arrangement of components and sensors in FIG. 8 demonstrates a robust and efficient system for filtering water, which can be necessary for safe and effective dialysis therapy. If the controller 104 determines that the water does not meet the required standards for dialysis treatment, the controller 104 can divert the fluid via valve 188 (FIG. 2), before providing it to the distribution system 154. In some embodiments, the fluid can be diverted by the controller 104, using valve 188, to a drain. In some embodiments, the fluid can be recirculated to a specific portion of the treatment system 152 based on the data received from the fluid at the sampling point 814. For example, based on the fluid tested at the sampling point 814, the controller 104 can determine that the fluid contains a concentrate that needs to be recirculated through the ultrafilter 204. In some embodiments, the fluid may need to be recirculated through the treatment system 152 rather than a targeted delivery to a component (e.g., the ultrafilter 204) therein.

FIG. 9 shows a detailed schematic of a distribution system for a water purification system, highlighting the integration and interaction of various components to ensure the production of high-purity water suitable for dialysis treatments. This figure builds upon the features depicted in FIG. 5, introducing additional components and sensors that can be used in the delivery of dialysis fluid constituents.

In some embodiments, the distribution loop can include the fluid reservoir 300 and the pump 302 that is configured to circulate the water within the distribution system 154 to prevent stagnation. In some embodiments, the distribution system 154 includes a UV lamp 304. In some embodiments, the UV lamp 304 can prevent bacterial formation. In some embodiments, the ultrafilter 190 (FIG. 2) is configured to be located downstream of the distribution system 154 and upstream of the proportioning system 106, the cycler 112 (FIG. 1), or both, to remove endotoxins produced by the UV lamp 304. A sampling point 902 can be included, in some embodiments, downstream of the fluid reservoir 300, allowing for periodic testing and verification of the water quality at this stage. In some embodiments, the pump 302 can be a gear pump configured for purified water delivery.

While the pump 302 is responsible for circulating the water through at least the distribution system 154, the distribution system 154 can, in some embodiments, include the flowrate sensor 310 to measure the flow rate at which the water is pumped by the pump 302. In some embodiments, the flowrate sensor 310 can be in electronical communication with the controller 104. Using the data from the flowrate sensor 310, the controller 104 can control the pump 302 to adjust the flowrate of the fluid from the fluid reservoir 300.

The distribution system 154 can include a conductivity sensor 904 to provide real-time monitoring of the conductivity of the water. In some embodiments, this can provide the controller 104 with information regarding the ionic content of the water which can provide an indication of dissolved salts and impurities.

The distribution system 154 can include a temperature sensor 906 to monitor the water temperature. The temperature sensor 906 can provide the controller 104 with information regarding the temperature of the fluid exiting the fluid reservoir 300. In some embodiments, the conductivity sensor 904 and the temperature sensor 906 can be combined as a single component. In some embodiments, the conductivity sensor 904 and the temperature sensor 906 can be a single sensor capable of measuring the electrical conductivity of the solution along with the temperature of the fluid downstream from the fluid reservoir 300 and the pump 302.

In some embodiments, if the temperature or conductivity from the temperature sensor 906 or conductivity sensor 904, respectively, is outside of a predetermined threshold, the controller 104 can use a valve 908 to divert the fluid. For example, in some embodiments, the controller 104 can divert the fluid, using the valve 908, to the drain to flush the fluid until the fluid is within the predetermined thresholds.

In some embodiments, the distribution system 154 can include the pressure sensor 318 to monitor the pressure within the system. The valve 320 provides additional control over the water flow, directing the flow towards the ultrafilter 190 or through a recirculation loop 914, depending on the system's requirements. In some embodiments, the recirculation loop 914 is fluidly connected to the fluid reservoir 300 to permit fluid control to recirculate through at least a portion of the distribution system 154. In some embodiments, the recirculation loop 914 can be used, by the controller 104, the prevent water stagnation when the preparator 114 is not producing dialysis fluid. For example, when sufficient fluid has been generated for a dialysis therapy at a specific point during the therapy.

In some embodiments, the recirculation loop 914 can include the UV lamp 304. For example, the UV lamp 304 can prevent bacterial formation while water is circulating in the recirculation loop 914.

In some embodiments, the distribution system 154 can include the ultrafilter 190 to remove particulates and ensure the water is free from endotoxins and other contaminants.

In some embodiments, a second sampling point 912 is included downstream of the ultrafilter 190, facilitating additional testing and verification of the water quality before the water is used in dialysis treatments. The valve 910, located downstream of the ultrafilter 190, offers additional control over the fluid flow. In some embodiments, the valve 910 can be controlled by the controller 104 to divert water from the fluid line based on the data received from the second sampling point 912 before the fluid is delivered to the proportioning system 106.

In reference to FIG. 10, the proportioning system 106 can include, in some embodiments, a heat exchanger 1002 that can be configured to receive water from the distribution system 154. In some embodiments, the heat exchanger 1002 can be a heating element configured to heat the fluid accommodated by the heat exchanger 1002, traveling through the heat exchanger 1002, or any combination thereof. In some embodiments, the heat exchanger 1002 is not included.

In some embodiments, upstream from the heat exchanger 1002, the proportioning system 106 can include the dialysis fluid concentrate source 350 and the dialysis fluid concentrate source 352. In some embodiments the dialysis fluid concentrate source 350 and the dialysis fluid concentrate source 352 can be pre-mixed in a single container. For example, the dialysis fluid concentrate source 350 and the dialysis fluid concentrate source 352 can be delivered to the patient in a pre-mixed form which can be used to create a dialysis solution rather than being delivered in two separate containers. In some embodiments, the dialysis fluid concentrate source 350 can include an ion solution. In some embodiments, the dialysis fluid concentrate source 352 can include a dextrose solution. In some embodiments, a third dialysis fluid concentrate source can be included. In such embodiments, the third dialysis fluid concentrate source can include sodium bicarbonate. In some embodiments, the bicarbonate can be combined with either the dialysis fluid concentrate source 350 or the dialysis fluid concentrate source 352. In some embodiments, the dialysis fluid concentrate source 350 can be in a powder form. In some embodiments, the dialysis fluid concentrate source 350 can be in a liquid form. In some embodiments, the dialysis fluid concentrate source 352 can be in a powder form. In some embodiments, the dialysis fluid concentrate source 352 can be in a liquid form. In embodiments in which the dialysis fluid concentrate source 350 or the dialysis fluid concentrate source 352 are in powder form, water can be added to the dialysis fluid concentrate source 350 or the dialysis fluid concentrate source 352 in powder form via the water source 354 prior to being utilized by the proportioning system 106. In some embodiments, the water source 354 can be the water purification system 102 (FIG. 1). For example, the water source 354 can be the fluid reservoir 300.

In some embodiments, the controller 104 can control a valve 1004 fluidly connected to the dialysis fluid concentrate source 350 to introduce a dialysis fluid concentrate accommodated in the dialysis fluid concentrate source 350 into a fluid conduit 1022. Similarly, in some embodiments, the controller 104 can control a valve 1010 fluidly connected to the dialysis fluid concentrate source 352 to introduce a dialysis fluid accommodated in the dialysis fluid concentrate source 352 into a fluid conduit 1022.

In some embodiments, the proportioning system 106 can include a plurality of scales to measure the weight of the dialysis fluid concentrate source 350, the dialysis fluid concentrate source 352, and a mixing container 1032. The plurality of scales can include, in some embodiments, a scale 1006 configured to weigh the dialysis fluid concentrate source 350. In some embodiments, the scale 1006 can be electronically connected to the controller 104 to provide the weight of the dialysis fluid concentrate source 350. The weight provided by the scale 1006 can allow the controller 104 to determine the weight of the dialysis fluid concentrate source 350. As the dialysis fluid is delivered from the dialysis fluid concentrate source 350, a new weight can be provided to the controller 104. Using the new weight, in some embodiments, the controller 104 can calculate how much dialysis fluid from the dialysis fluid concentrate source 350 has been used based on the change in weight. For example, the controller 104 can calculate the change in weight from the dialysis fluid concentrate source 350 to determine how much of the dialysis fluid has been used from the dialysis fluid concentrate source 350. In some embodiments, the weight of the dialysis fluid concentrate source 350, the dialysis fluid concentrate source 352, and the container 360 are measured real-time.

Similarly, the plurality of scales can include, in some embodiments, a scale 1008 configured to weigh the dialysis fluid concentrate source 352. In some embodiments, the scale 1008 can be electronically connected to the controller 104 to provide the weight of the dialysis fluid concentrate source 352. The weight provided by the scale 1008 can allow the controller 104 to determine the weight of the dialysis fluid concentrate source 352. As the dialysis fluid is delivered from the dialysis fluid concentrate source 352, a new weight can be provided to the controller 104. Using the new weight, in some embodiments, the controller 104 can calculate how much dialysis fluid from the dialysis fluid concentrate source 352 has been used based on the change in weight. For example, the controller 104 can calculate the change in weight from the dialysis fluid concentrate source 352 to determine how much of the dialysis fluid has been used from the dialysis fluid concentrate source 352. In some embodiments, the plurality of scales 1006, 1008, and 1034 can indicate to the controller 104 when a target delivery weight has been reached. For example, in some embodiments, the target delivery weight can be when the scale measurement indicates a certain change in weight on the respective scale of the plurality of scales. In some embodiments, the target delivery weight can be from the scale 1034 configured to measure the change in weight of the mixing container 1032.

Additionally, the plurality of scales can include, in some embodiments, a scale 1034 configured to weigh a mixing container 1032. In some embodiments, the scale 1034 can communicate data to the controller 104 to provide the weight of the mixing container 1032. The weight provided by the scale 1034 can allow the controller 104 to determine the weight of the mixing container 1032. As the fluid is delivered from the dialysis fluid concentrate source 350, the dialysis fluid concentrate source 352, and water from the distribution system 154, to the mixing container 1032, a new weight of the mixing container 1032 can be provided to the controller 104.

In some embodiments, the controller 104 can use the data from the scale 1006, the scale 1008, the scale 1034, or any combination thereof to determine at least the concentration of concentrates within the dialysis fluid in the mixing container 1032 based on the change in weight from the dialysis fluid concentrate source 350 and the dialysis fluid concentrate source 352. For example, if the weight of dialysis fluid concentrate source 350 decreases by 350g, the controller 104 can calculate the percentage of the dialysis fluid from the dialysis fluid concentrate source 350 disposed within the mixing container 1032. In some embodiments, the controller 104 can calculate the composition of the dialysis fluid within the mixing container 1032 by measuring the weight of only the scale 1034.

In some embodiments, the dialysis fluid concentrate source 350 and the dialysis fluid concentrate source 352 can be stored in a container such as, but not limited to, a bag having fluid connections for fluidly connecting to the fluid conduit 1022. In some embodiments, the fluid conduit 1022 is configured to receive water from the distribution system 154, dialysis fluid from the dialysis fluid concentrate source 350, and dialysis fluid from the dialysis fluid concentrate source 352. In some embodiments, the fluid conduit 1022 is configured to receive the water and the dialysis fluid concentrates at the same time. In some embodiments, the fluid conduit 1022 is configured to receive the water and the dialysis fluid concentrates at separate points in time. For example, in some embodiments, the fluid conduit 1022 can receive each of the dialysis fluids and the water consecutively. In some embodiments, the fluid conduit 1022 is configured to receive the dialysis fluid concentrate from the dialysis fluid from the dialysis fluid concentrate source 350 at a first point in time, the dialysis fluid from the dialysis fluid concentrate source 352 at a second point in time, and the water from the distribution system 154 at a third point in time. In some embodiments, the order which the fluid conduit 1022 receives the dialysis fluid from the dialysis fluid concentrate source 350 and the dialysis fluid concentrate source 352 can be adjusted by the controller 104 based on data received from the various sensors within the system.

In some embodiments, the controller 104 can control the distribution of the dialysis fluid from the dialysis fluid concentrate source 350, the dialysis fluid from the dialysis fluid concentrate source 352, and the water from the distribution system 154 in a plurality of phases. For example, in a first phase of the plurality of phases, the controller 104 can introduce the dialysis fluid from the dialysis fluid concentrate source 350, the dialysis fluid from the dialysis fluid concentrate source 352, and the water from the distribution system 154 simultaneously. In a second phase of the plurality of phases, the controller 104 can introduce dialysis fluid concentrate and water sequentially until the desired amount of dialysis fluid can be created.

In some embodiments, the controller 104 can control the valves 1004, 1010 to introduce dialysis fluid from the dialysis fluid concentrate source 350 and the dialysis fluid concentrate source 352 in a first phase of the plurality of phases. In some embodiments, the first phase of the plurality of phases includes introducing water from the distribution system 154 In a second phase of the plurality of phases, water from the distribution system 154 can be delivered to the fluid conduit 1022. The second phase of the plurality of phases, in some embodiments, can flush the dialysis fluid remaining in the fluid conduit 1022 from the first phase, to ensure that the patient receives the desired concentration of dialysis fluid. The water from the distribution system 154 can carry the dialysis fluid from the concentrate sources 350, 352 to the mixing container 1032, ensuring the prescribed dialysis solution is created with the desired concentrate mixture.

In some embodiments, the plurality of phases can include a third phase, where the controller 104 can control the introduction of the concentrates from the dialysis fluid concentrate source 350 and the dialysis fluid concentrate source 352, along with the introduction of water from the distribution system 154, at discrete and separate intervals. For example, in some embodiments, sequentially introducing one fluid at a time.

In some embodiments, the first phase of the plurality of phases can be the procedure described in FIG. 7. In some embodiments, the controller 104 delivers water as a final fluid into the mixing container 1032. By using water as the final fluid, the dialysis fluid within the mixing container 1032 can better achieve batch homogeneity.

In some embodiments, the fluid conduit 1022 can have the static mixer 358 (FIG. 6) within the fluid conduit 1022. In some embodiments, the fluid conduit 1022 can include a plurality of the static mixer 358. For example, in some embodiments, static mixers can be included at junctions of each of the dialysis fluid concentrate conduits with the fluid conduit 1022. The static mixer 358 can be a fixed device that is installed within the fluid conduit 1022 to allow for the blending of the water and the dialysis fluid concentrates as they move through the fluid conduit 1022. In some embodiments, the fluid conduit 1022 can be the mixing conduit 356.

It is to be appreciated that the dialysis fluid concentrate source 350 and the dialysis fluid concentrate source 352 being separate is an example. For example, in some embodiments, the dialysis fluid concentrate source 350 and the dialysis fluid concentrate source 352 can be combined into the same container. In some embodiments, the dialysis fluid from the dialysis fluid concentrate source 350 and the dialysis fluid from the dialysis fluid concentrate source 352 can be pre-mixed into a single container by the patient, the manufacturer, the clinician, or any combination thereof, before being connected to the dialysis system 100.

In some embodiments, the proportioning system 106 can include a pump 1012 configured to transfer fluid throughout the fluid conduit 1022. For example, the pump 1012 can be fluidly connected to the fluid conduit 1022. The pump 1012 can be configured to communicate with the controller 104 and be controlled by the controller 104. For example, the controller 104 can adjust the operating speed of the pump 1012 based on: the data from the plurality of scales 1006, 1008, 1034; the various sensors throughout the dialysis system 100; data collected from the various sampling points disposed in the dialysis system 100; or any combination thereof. In some embodiments, the pump 1012 can be a piston pump configured to delivery the concentrates in dialysis fluid concentrate source 350 and the dialysis fluid concentrate source 352. In some embodiments, the pump 1012 can be a volumetric pump configured to determine the flow rate of the fluid from the dialysis fluid concentrate source 350 and the dialysis fluid concentrate source 352 by having certain pump stroke and rotational speed (RPM). In some embodiments, the pump 1012 can be a volumetric pump configured to determine the flow rate of the fluid from the dialysis fluid concentrate source 350 and the dialysis fluid concentrate source 352 by having the controller 104 control the pump stroke and rotational speed at specified values.

In some embodiments, the proportioning system 106 can include a valve 1018 connected to a pump bypass circuit 1020. In some embodiments, by controlling the valve 1018 to divert the fluid into the pump bypass circuit 1020, the fluid within fluid conduit 1022 can flow around pump 1012, enabling flow rates that are unimpeded by the pump 1012. For example, the pump 1012 can be an occlusive piston pump, in some embodiments, which can cause flow rates of the fluid within fluid conduit 1022 to slow down. For example, the fluid may have sufficient velocity, from the pump 302, to travel at a desired flow rate until the fluid within the fluid conduit 1022 reaches the mixing container 1032. In some embodiments, by diverting the fluid around the pump 1012 via the pump bypass circuit 1020, the fluid can flow without any dampening effect that may exist from the pump 1012. Additionally, by bypassing the pump 1012, the controller may not need to use pump 1012 to deliver fluid from the fluid conduit 1022 to the mixing container 1032, enabling a more efficient, less noisy, dialysis fluid generation process.

In some embodiments, the proportioning system 106 can include a sampling point 1014 after downstream from the pump 1012 to test the fluid within the fluid conduit 1022 at this junction. In some embodiments, the sampling point 1014 can be fluidly downstream from the pump bypass circuit 1020 to permit testing of the fluid after recirculation. In some embodiments, the sampling point 1014 can be upstream from an entry to the pump bypass circuit 1020 to permit testing of the fluid, by the controller 104, as the fluid is circulated through the pump bypass circuit 1020. For example, the controller 104 can test the fluid being recirculated and make a determination if the fluid should proceed to the mixing container 1032 or be diverted to a drain 1027. In some embodiments, the drain 1027 can be the same drain as 220. In some embodiments, the drain 1027 can be fluidly connected to the drain 220. In some embodiments, the drain 1027 can be fluidly connected to a drain reservoir. This can be advantageous as the controller 104 calculates the weight of the dialysis fluid concentrate source 350, the weight of the dialysis fluid concentrate source 352, and the weight of the mixing container 1032 to determine the composition of the dialysis fluid within the mixing container 1032.

In some embodiments, the proportioning system 106 can include a conductivity sensor 1016, a pressure sensor 1023, and a temperature sensor 1024 between the pump 1012 and the mixing container 1032. The conductivity sensor 1016, the pressure sensor 1023, and the temperature sensor 1024 can, in some embodiments, be configured to communicate with the controller 104 and provide data to the controller 104. The conductivity sensor 1016, in some embodiments, can measure the electrical conductivity of the fluid within the fluid conduit 1022, which can be used by the controller 104 to determine a concentration of ions in the fluid. The pressure sensor 1023 can measure the pressure of the fluid within the fluid conduit 1022, which can be used by the controller 104 to determine properties of the dialysis system 100 to adjust, for example, the operation speed of the pump 1012. In some embodiments, the conductivity sensor 1016, the pressure sensor 1023, and the temperature sensor 1024 can allow the controller 104 to check hydraulic parameters and composition of the fluids disposed in the fluid conduit 1022 to prevent, or detect, a hazardous situation. If the fluid in the fluid conduit 1022 indicates an unknown composition of fluid, a parameter that may be unexpected, or indicates that the proportioning system 106 is operating outside acceptable thresholds, the controller 104 can obtain this information from the sensors 1016, 1023, and 1024.

In some embodiments, the proportioning system 106 can include a valve 1026 to permit control of the fluid within the fluid conduit 1022. In some embodiments, the valve 1026 can be downstream from the conductivity sensor 1016, the pressure sensor 1023, the temperature sensor 1024, or any combination thereof, to permit the controller 104 to obtain data from those sensors, analyze the data, and determine if the fluid should proceed through the valve 1026 to the mixing container 1032 or be diverted to a drain 1027. In some embodiments, the valve 1026 can be fluidly between the mixing container 1032 and the sampling point 1014. In some embodiments, the valve 1026 can be an electro-clamp that can prevent flow across the valve 1026. For example, the controller 104 can determine that a batch of peritoneal fluid has been produced and can stop flow to the mixing container 1032 at valve 1026. In some embodiments, the proportioning system 106 can include a clamp 1025 to occlude the flow path at this location. Having an additional clamp 1025 can prevent the need for valve 1026 to have added complexity, which can increase manufacture costs and create additional points of failure in the proportioning system 106.

In some embodiments, the dialysis system 100 can include a disposable kit 1028, or a disposable preparator kit, that can include a series of valves, filters, and flow paths to selectively distribute fluid to the cycler 112, the mixing container 1032, or any combination thereof. In some embodiments, the disposable kit 1028 is configured to filter the fluid that travels therethrough to ensure the sterility of the dialysis fluid produced.

In some embodiments, the proportioning system 106 can include the mixing container 1032 fluidly connected to the fluid conduit 1022. As described above, the controller 104 can operate the pump 1012, and respective valves, to direct fluid from the dialysis fluid concentrate source 350, the dialysis fluid concentrate source 352, or the distribution system 154 through the fluid conduit 1022 and into the mixing container 1032. The mixing container 1032 can be a container configured to accommodate a volume of dialysis fluid. In some embodiments, the mixing container 1032 can include elements that encourage mixing of various fluid disposed therein. For example, there may be moveable elements within the mixing container 1032 to facilitate mixing of the fluid disposed therein. Once the fluid is sufficiently mixed within the mixing container 1032, as can be determined by the controller 104 in some embodiments, the dialysis fluid can be delivered to the cycler 112 for heating and/or distribution to the patient.

FIG. 11 is a flowchart of an example method 1100 preparing dialysis fluid using a proportioning system (e.g., the proportioning system 106 of FIG. 10), according to some embodiments.

In some embodiments, at block 1102, the method 1100 includes receiving, in a mixing container (e.g., mixing container 1032 of FIG. 10), a first dialysis fluid concentrate. In some embodiments, an amount of the first dialysis fluid concentrate is determined by a weight of at least one of: a first dialysis fluid concentrate source 350; the mixing container 1032; or any combination thereof. In some embodiments, the weight is measured by at least one scale (e.g., the plurality of scales of FIG. 10) configured to measure the weight of the dialysis fluid concentrate source 350, the mixing container 1032, or any combination thereof.

In some embodiments, at block 1104, the method 1100 includes receiving in the mixing container 1032, a second dialysis fluid concentrate. In some embodiments, an amount of the second dialysis fluid concentrate is determined by a weight of at least one of a dialysis fluid concentrate source 352; the mixing container 1032; or any combination thereof. In some embodiments, the weight is measured by at least one scale configured to measure the weight of the dialysis fluid concentrate source 352, the mixing container 1032, or any combination thereof.

In some embodiments, at block 1106, the method 1100 includes receiving, in the mixing container 1032, water. In some embodiments, an amount of the water is determined by the weight of the mixing container 1032. In some embodiments, the weight of the mixing container 1032 is measured by the at least one scale. In some embodiments, the water is purified.

FIG. 12 is a flowchart of an example method 1200 preparing dialysis fluid using a proportioning system (e.g., the proportioning system 106 of FIG. 10), according to some embodiments.

In some embodiments, at block 1202, the method 1200 includes receiving, in a mixing container (e.g., mixing container 1032 of FIG. 10) a first dialysis fluid concentrate and a second dialysis fluid concentrate from at least one fluid concentrate source 350, 352. In some embodiments, at least an amount of the first dialysis fluid concentrate and the second dialysis fluid concentrate are received by the mixing container simultaneously. For example, the fluid conduit 1022 can accommodate a first dialysis fluid concentrate from the dialysis fluid concentrate source 350 and, at the same time, accommodate a second dialysis fluid concentrate from the dialysis fluid concentrate source 352. The pump 1012 can act on this fluid within the fluid conduit 1022 to deliver it to the mixing container 1032. In some embodiments, the method 1200 includes an amount of the first dialysis fluid concentrate that is received by the mixing container can be determined by a change in weight of at least one of: a first dialysis fluid concentrate source; the mixing container; or any combination thereof. In some embodiments, the method 1200 includes an amount of the second dialysis fluid concentrate received by the mixing container can be determined by a change in weight of at least one of: a second dialysis fluid concentrate source; the mixing container; or any combination thereof.

In some embodiments, at block 1204, the method 1200 includes receiving, in the mixing container 1032, water. In some embodiments, the water can be purified water from the distribution system 154. In some embodiments, the water can be from the fluid reservoir 300. In some embodiments, the method 1200 includes that an amount of the water received by the mixing container can be at a period of time subsequent to the mixing container receiving the first dialysis fluid concentrate and the second dialysis fluid concentrate. For example, in some embodiments, a first phase of the method 1200 can include delivering the dialysis fluid concentrate from the dialysis fluid concentrate source 350 and the dialysis fluid concentrate from the dialysis fluid concentrate source 352 to the mixing container 1032. Then, in a second phase, the method 1200 can include delivering water to the mixing container 1032. In some embodiments, having the water delivered subsequent to the dialysis fluid concentrates can allow the water to pick up any residual dialysis fluid concentrate left in the fluid conduit 1022. That can permit the dialysis system 100 to have a more accurate measure on how much dialysis fluid concentrate resides within the mixing container 1032, and thus, the prepared dialysis fluid. In some embodiments, the method 1200 can include that an amount of the water received by the mixing container is determined by a change in weight of the mixing container.

The terminology used herein is intended to describe embodiments and is not intended to be limiting. The terms “a,” “an,” and “the” include the plural forms as well, unless clearly indicated otherwise. The terms “comprises” and/or “comprising,” when used in this Specification, specify the presence of the stated features, integers, steps, operations, elements, and/or components, but do not preclude the presence or addition of one or more other features, integers, steps, operations, elements, and/or components.

It is to be understood that changes may be made in detail, especially in matters of the construction materials employed and the shape, size, and arrangement of parts without departing from the scope of the present disclosure. This Specification and the embodiments described are examples, with the true scope and spirit of the disclosure being indicated by the claims that follow.

What is claimed is:

1. A system comprising:
a fluid conduit configured to receive water from a water source, a first dialysis fluid concentrate, and a second dialysis fluid concentrate;
a pump fluidly connected to the fluid conduit and configured to pump the first dialysis fluid concentrate and the second dialysis fluid concentrate;
a first dialysis fluid concentrate source fluidly connected to the pump and configured to store the first dialysis fluid concentrate;
a second dialysis fluid concentrate source fluidly connected to the pump and configured to store the second dialysis fluid concentrate;
a processor configured to:
control an amount of mixing of the first dialysis fluid concentrate, the second dialysis fluid concentrate, and the water to output a dialysis fluid having a defined composition,
wherein the processor determines the defined composition of the dialysis fluid based on a change in weight of:
the first dialysis fluid concentrate source;
the second dialysis fluid concentrate source;
a mixing container configured to accommodate the dialysis fluid;
or any combination thereof.

2. The system of claim 1, wherein the first dialysis fluid concentrate is a liquid concentrate.

3. The system of claim 1, wherein the second dialysis fluid concentrate is a liquid concentrate.

4. The system of claim 1, wherein the first dialysis fluid concentrate source is fluidly connected to a water source to receive water to be added to the first dialysis fluid concentrate source.

5. The system of claim 1, wherein the second dialysis fluid concentrate source is fluidly connected to a water source to receive water to be added to the second dialysis fluid concentrate source.

6. The system of claim 1, further comprising:
a first sensor configured to determine a first amount of the first dialysis fluid concentrate in the first dialysis fluid concentrate source; and
a second sensor configured to determine a second amount of the second dialysis fluid concentrate in the second dialysis fluid concentrate source.

7. The system of claim 6, wherein at least one of the first sensor or the second sensor is a scale configured to measure a weight.

8. The system of claim 1, further comprising a fluid outlet, wherein the fluid outlet is configured to receive the dialysis fluid from the mixing container.

9. The system of claim 8, further comprising a sensor upstream of the fluid outlet, wherein the sensor is configured to determine whether the dialysis fluid meets the defined composition.

10. A method for preparing a dialysis fluid, comprising:
receiving, in a mixing container, a first dialysis fluid concentrate, wherein, an amount of the first dialysis fluid concentrate received by the mixing container is determined by a change in weight of at least one of: a first dialysis fluid concentrate source; the mixing container; or any combination thereof; and
receiving in the mixing container, a second dialysis fluid concentrate, wherein an amount of the second dialysis fluid concentrate received by the mixing container is determined by a change in weight of at least one of: a second dialysis fluid concentrate source; the mixing container; or any combination thereof,
receiving, in the mixing container, water,
wherein an amount of the water received by the mixing container is determined by a change in weight of at least one of: the mixing container; the water source container, or any combination thereof.

11. The method of claim 10, wherein the change in weight of at least one of: the first dialysis fluid concentrate source; the mixing container; or any combination thereof, is measured by at least one scale configured to measure the weight of the dialysis fluid concentrate source, the mixing container, or any combination thereof.

12. The method of claim 10, wherein the change in weight of at least one of: the second dialysis fluid concentrate source; the mixing container; or any combination thereof, is measured by at least one scale configured to measure the weight of the second fluid concentrate source, the mixing container, or any combination thereof.

13. The method of claim 10, wherein the change in weight of the mixing container is measured by at least one scale configured to measure the weight of the mixing container.

14. The method of claim 10, the method comprising:
determining, by a controller, a composition of a fluid within the mixing container.

15. The method of claim 14, wherein, in the determining, the controller is configured to determine the composition of the fluid within the mixing container based on a change in weight of at least one of:
the first dialysis fluid concentrate source;
the second dialysis fluid concentrate source;
the mixing container; or
any combination thereof.

16. A method for preparing a dialysis fluid, comprising:
receiving, in a mixing container, a first dialysis fluid concentrate and a second dialysis fluid concentrate from at least one fluid concentrate source,
wherein at least an amount of the first dialysis fluid concentrate and the second dialysis fluid concentrate source are received by the mixing container simultaneously;
wherein an amount of the first dialysis fluid concentrate received by the mixing container is determined by a change in weight of at least one of: a first dialysis fluid concentrate source; the mixing container; or any combination thereof, and
wherein an amount of the second dialysis fluid concentrate received by the mixing container is determined by a change in weight of at least one of: a second dialysis fluid concentrate source; the mixing container; or any combination thereof; and
receiving, in the mixing container, water,
wherein an amount of the water received by the mixing container is at a period of time subsequent to the mixing container receiving the first dialysis fluid concentrate and the second dialysis fluid concentrate;
wherein an amount of the water received by the mixing container is determined by a change in weight of the mixing container.

17. The method of claim 16, wherein the change in weight of at least one of: the first dialysis fluid concentrate source; the mixing container; or any combination thereof, is measured by at least one scale configured to measure the weight of the dialysis fluid concentrate source, the mixing container, or any combination thereof.

18. The method of claim 16, wherein the change in weight of at least one of: the second dialysis fluid concentrate source; the mixing container; or any combination thereof, is measured by at least one scale configured to measure the weight of the second fluid concentrate source, the mixing container, or any combination thereof.

19. The method of claim 16, the method comprising:
determining, by a controller, a composition of a fluid within the mixing container.

20. The method of claim 19, wherein, in the determining, the controller is configured to determine the composition of the fluid within the mixing container based on a change in weight of at least one of:
the first dialysis fluid concentrate source;
the second dialysis fluid concentrate source;
the mixing container; or
any combination thereof.
